(19) United States
(12) Patent Application Publication
(10) Pub. No.: US 2011/0184298 A1
(43) Pub. Date: Jul. 28, 2011

(54) PORTABLE CARDIO WAVEFORM ACQUISITON AND HEART RATE VARIABILITY (HRV) ANALYSIS

(75) Inventors: Eduardo De Marchena, Miami, FL (US); Suresh Atapattu, Platation, FL (US)

(73) Assignee: University of Miami, Miami, FL (US)

(21) Appl. No.: 13/121,132

(22) PCT Filed: Sep. 25, 2009
(86) PCT No.: PCT/US2009/058313

§ 371 Date: Mar. 25, 2011

Related U.S. Application Data
(60) Provisional application No. 61/099,939, filed on Sep. 25, 2008.

(51) Int. Cl. A61B 5/0402

(52) U.S. Cl. 600/509

(57) ABSTRACT

A system and method for measuring heart rate variability (HRV). The system includes at least one biosensor operable to measure at least one signal from the heart. A bioamplifier is also included, the bioamplifier is in communication with the at least one biosensor. The bioamplifier amplifies the at least one signal into at least one amplified analog signal. A portable device is included, the portable device is in communication with the bioamplifier and operable to digitize the at least one amplified signal into one or more digital signals and measure inter-beat intervals from the one or more digital signals and calculate HRV from the measured inter-beat intervals. A database is also included in communication with the portable device, wherein the measured HRV is indexed in the database by one or more criteria.

### CROSS-REFERENCE TO RELATED APPLICATION

This application is 35 U.S.C §371 of International Application No. PCT/US09/58313, filed Sep. 25, 2009, entitled PORTABLE CARDIO WAVEFORM ACQUISITION AND HEART RATE VARIABILITY (HRV) ANALYSIS, which application claims priority to U.S. Provisional Patent 61/099939, filed Sep. 25, 2008, the entirety of which is incorporated herein by reference.

### STATEMENT REGARDING FEDERALLY SPONSORED RESEARCH OR DEVELOPMENT

n/a

### FIELD OF THE INVENTION

The present invention relates to a method and system for acquiring various cardiac waveforms and determining real-time heart rate variability with a portable medical device.

### BACKGROUND OF THE INVENTION

The autonomic nervous system plays a role in a wide range of somatic and mental diseases. Scientific research has shown how autonomic imbalance and decreased parasympathetic tone, in particular, may be the final common pathway linking negative affective states and conditions to ill health. Assessment of heart rate variability (HRV) has been recognized as a non-invasive means of evaluating cardiac autonomic tone. Previous studies on the effects of pharmacological blockade or physiological manipualition of autonomic influences have suggested that measures of HRV are reflective of the level of sympathetic or parasympathetic activity. HRV is also regarded as an indicator of the activity of autonomic regulation of circulatory function.

Additionally, there is a significant relationship between the autonomic nervous system and cardiovascular mortality. Experimental evidence has shown an association between lethal cardiac arrythmias and increased sympathetic or reduced vagal activity. Thus, the association between impaired cardiac autonomic tone and propensity to lethal arrythmias makes assessment of cardiac balance, as obtained from HRV, of particular practical importance.

The principal aim of HRV research is to obtain the necessary information to predict susceptibility to Sudden Cardiac Death (SCD). In the United States, it is estimated that there are 300,000-460,000 deaths due to SCD; hence the great interest in HRV. Published work has shown that HRV is good indicator of well being. Low HRV is associated with morbidity, while high HRV is associated with wellness.

A known method of calculating HRV involves the use of a pulse oximeter. However, the HRV calculated from the use of pulse oximeter can be inaccurate due to disease, anatomical variations, and lack of precision. For example, HRV requires normal sinus rhythm that cannot be fully validated with only pulse rate recording. Other methods to calculate HRV have included using a dry contact electrode for electrocardiogram (ECG) recording from one hand of a patient. However, ECG electrodes are typically coupled to cumbersome and large equipment and often require long intervals of recording to determine an accurate HRV.

Therefore, a need exists for an HRV data acquisition and analysis process to be fast, reliable, easy to use, and portable. Also, the process must obtain reproducible results and not interfere with clinical operations.

### SUMMARY OF THE INVENTION

The present invention advantageously provides a system for acquiring various cardiac waveforms and determining real-time heart rate variability with a portable device. The system includes at least one biosensor operable to measure at least one signal from the heart. A bioamplifier is included in communication with the at least one biosensor. The bioamplifier amplifies the at least one signal into at least one amplified signal. A portable device is included in communication with the bioamplifier. The portable device is further in communication with a analog to digital converter operable to digitize the at least one amplified signal into one or more digital signals. The portable device further measures the inter-beat intervals from the one or more digital signals and calculates HRV from the measured inter-beat intervals. A database is also included in communication with the portable device, wherein the measured HRV is indexed in the database by one or more criteria.

In another embodiment of the present invention, the method includes measuring at least one signal proximate the heart. The at least one signal is then amplified into at least one amplified signal. The at least one amplified signal is then digitized into one or more digital signals. From the one or more digitized signals the inter-beat intervals are measured and HRV is calculated from the measured inter-beat intervals. The measured inter-beat intervals are then correlated to the calculated HRV to a condition of cardiovascular health.

In yet another embodiment of the present invention, the method includes providing a first biosensor operable to measure electrical activity proximate the heart. A second biosensor is also provided, the second biosensor being operable to measure changes in volume proximate the heart. A bioamplifier is also provided in communication with the first biosensor and the second biosensor. A portable device is provided in communication with the bioamplifier and operable to measure the inter-beat intervals and calculate HRV from the measured inter-beat intervals. The first biosensor and the second biosensor are then positioned proximate the heart. A first signal acquired from the first biosensor is measured and a second signal acquired from the second biosensor is also measured. The first signal and the second signal are then amplified into a first amplified signal and a second amplified signal. The first amplified signal and the second amplified signal are then transmitted to the portable device. The first amplified signal is then digitized to a first digital signal and the second amplified signal is then digitized to a second digital signal. The inter-beat intervals from the first digital signal or the second digital signal are then measured. HRV is then measured from the inter-beat intervals. The measured inter-beat intervals are then correlation with the measured HRV to a condition of cardiovascular health. The HRV data is then indexed in a remote database by one or more criteria. In response to the measured HRV a treatment protocol is then created.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 shows a top view of the components of an HRV acquisition system in a wired configuration in accordance with the present invention;

FIG. 2 shows the HRV acquisition system shown in FIG. 1 with a wireless configuration with the analog to digital converter on the biosensor;

FIG. 3 shows a flow chart illustrating the HRV and HR dissociation methodology;

FIG. 4 shows correlation data between HRV and HR and a comparison of this data between healthy patients and patients with coronary disorders; and

FIG. 5 shows a flow chart of a method in accordance with the present invention.

### DETAILED DESCRIPTION OF THE INVENTION

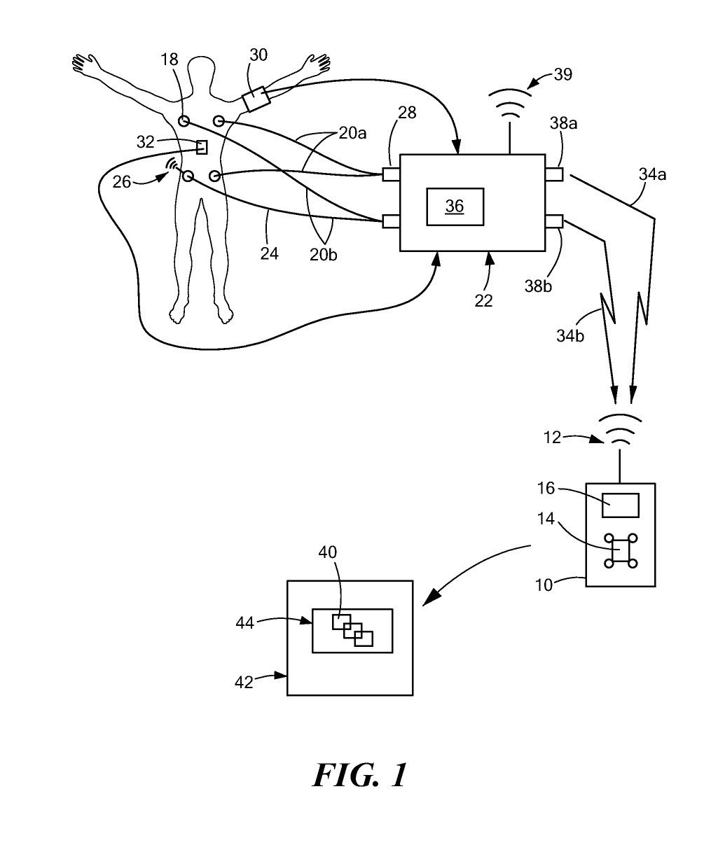

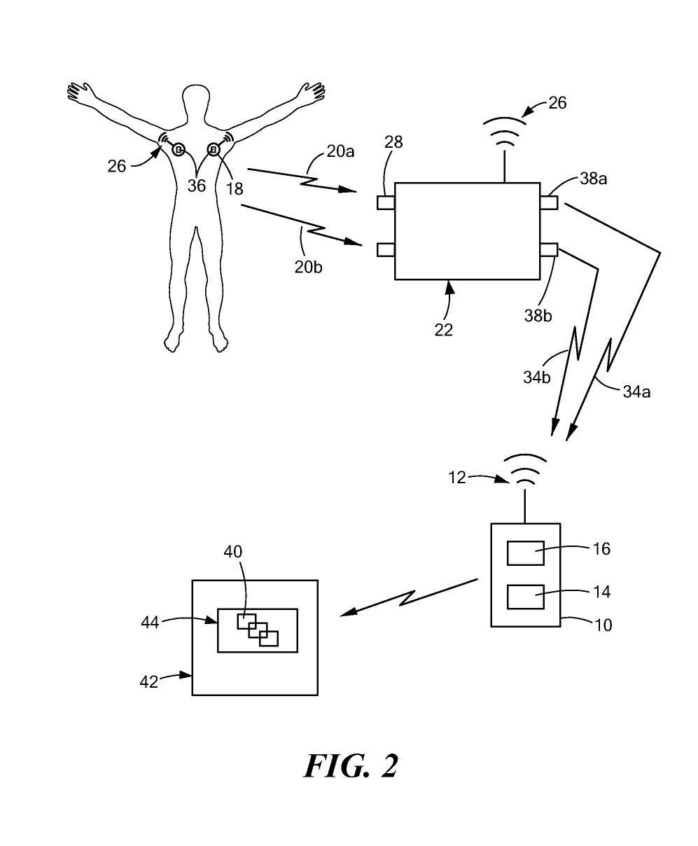

The present invention advantageously provides a system and method for acquiring various cardiac waveform signals and determining real-time heart rate variability with a portable device. Referring now to FIG. 1 and FIG. 2, wherein the various components of the present invention are illustrated. The HRV acquisition system includes a device 10, which may be a portable handheld device such as a PDA, smart phone, or small computer such as a laptop computer. A first wireless transceiver 12 may be included within the device 10 for wireles sly receiving and transmitting desired data. The device 10 may further include a microprocessor 14 operable to perform analog to digital signal conversions. A display 16 may also be included on the device 10, which may allow a user to read and interpret the various signals retrieved, recorded, and analyzed by the HRV acquisition system.

Continuing to refer to FIGS. 1 and 2, at least one biosensor 18 may be included in the HRV acquisition. The at least one biosensor 18 may detect, acquire, and measure at least one signal 20, which may be analog, for example, electrical, electromagnetic, acoustical, optical, or vibrational signals emitted from the heart or surrounding tissue of a patient. For example, the at least one biosensor 18 may include a photodetector and an emitter for photoplethysmography (PPG). PPG is an optically obtained plethysmograph, which is a volumetric measurement of an organ, for example, the heart. The at least one biosensor 18 may further include electrodes and transducers for electrocardiogram (ECG) detection. The electrodes may be in communication with the patent by conductive gel or non-gel based contact surfaces. In an embodiment, twelve electrodes are used for ECG detection to map and measure electrical activity from various locations on the patient's body. It is further contemplated that a combination of both the ECG and PPG biosensors 18 may be used detect various cardiac waveform analog signals 20, for example, a pulse waveform. Operators of the HRV system can selectively operate either the ECG or PPG biosensors 18, or both, from the controls on the device 10. Each of the at least one biosensor 18 may include its own internal power supply or alternatively be wired to an external power source.

The at least biosensor 18 may further be coupled to or otherwise in communication with a bioamplifier 22 by one or more lead wires 24 or connectors capable of transmitting the at least one signal 20 detected by the at least one biosensor 18. The bioamplifier 22 may alternatively be in communication with the at least one biosensor 18 through a second wireless transceiver 26 coupled to the at least one biosensor 18. For example, the at least one biosensor 18 may be in communication with the second wireless transceiver 26 and transmit the various detected, acquired, and measured at least one signal 20 (whether analog or digital) from the at least one biosensor 18 to the bioamplifier 22 or the device 10. One or more inputs 28 may also be included in the bioamplifier 22 for receiving the at least one signal 20 emitted from the second wireless transceiver 26.

A blood pressure monitor 30, for example a sphygmomanometer having a cuff, may be included in the HRV acquisition system in communication with the bioamplifier 22 and with the patient. The blood pressure monitor 30 may measure static blood pressure during the at least one signal 20 acquisition from the at least one biosensor 18. In addition, a respiratory rate sensor 32 may also be in included, the respiratory rate sensor 32 being in communication with the bioamplifier 22 to monitor the patient's breathing during the at least one signal 20 acquisition.

Continuing to refer to FIGS. 1 and 2. The bioamplifier 22 may include circuitry, such as a microprocessor, for signal recognition and amplification. In the embodiment shown in FIG. 1, the circuitry receives at least one signal 20 in analog form from the at least one biosensor 18 through the one or more inputs 28. The bioamplifier 22 may then amplify the at least one signal 20 by convolution, Fourier transform, or other methods known in the art, into one or more amplified signals 34. The bioamplifier 22 may be powered by an internal power source or may include an outlet for receiving external power. An analog to digital signal converter 36 may be included in the bioamplifier 22 (seen in FIG. 1), or the at least one biosensor 18 (seen in FIG. 2), to convert the at least one analog signal received into at least one or more digital signal 38 for analysis. As shown in FIG. 2, in an embodiment where the analog to digital converter 36 is disposed on the at least one biosensor 18, the at least one signal 20 may be digitally converted on the at least one biosensor 18 into the one or more digital signal 38. The one or more digital signal 38 may then be wirelessly transmitted from the at least one biosensor 18 to the bioamplifier 22 or to the device 10.

Referring back now to FIG. 1, the at least one amplified signal 34 may be processed and relayed to the device 10 by one or more outputs 38. Each output 38 may correspond to a different connection pathway for a particular at least one biosensor 18. For example, if there are three distinct at least one biosensors 18 that relay the at least one signal 20 to the bioamplifier 22, there may be three corresponding outputs 38 that relay each at least one amplified signal 34 from the bioamplifier 22 to the device 10.

Each output 38, which may relay a distinct amplified signal 34 to the device 10, may be selectively accessed by the device 10 depending on the desired analysis. For example, in an embodiment where a first signal 20a is an analog measurement of electrical activity and a second signal 20b is an analog measurement of volume, the bioamplifier 22 may amplify both the first signal 20a into a first amplified signal 34a and the second signal 20b into a second amplified signal 34b. The first amplified signal 34a may be relayed to a first output 38a and the second amplified signal 34b may be relayed to a second output 38b. The first output 38a and the second output 38b may further be selectively accessed by the device 10 through, for example, a third wireless transceiver 39 coupled to the bioamplifier 22 and in communication with the at least one biosensor 18 and the device 10, depending on the desired measurement, analysis, and signal (whether analog or digital) to be received. Alternatively, the bioamplifier 22 may in communication with the device 10 and the at least one biosensor 18 though wires.

Additional inputs 28 and outputs 38 may be included to relay blood pressure or respiratory rate information to the device 10 either directly in analog form or in digitized. It is further contemplated that the at least one signal 20 may be relayed to the device 10 while at least another signal 20 may be amplified and relayed to the device 10. The amplification and digitization of any or all of the at least one signal 20 or amplified signal 34 may be selectively operable by the device 10.

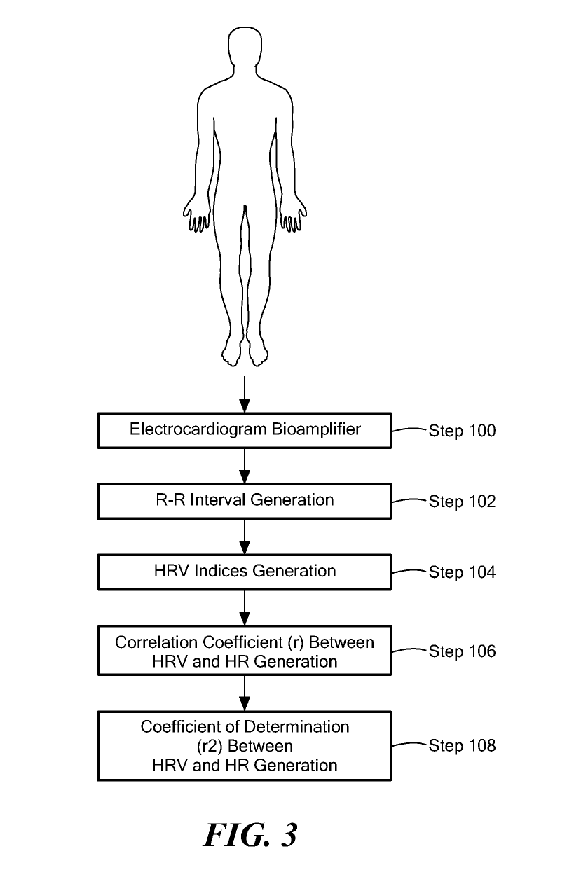

Now referring to FIG. 3, which shows a flow chart of the steps involved in measuring and recording HRV. The HRV acquisition method provides a computer readable medium residing within the device 10 that analyzes the various signals acquired via the bioamplifier 22, and may further display and interact with a user via a graphical user interface. The computer readable medium may further include an algorithm for interpreting, displaying, and transmitting the received analog or digital signals. For example, a real-time amplified ECG signal may be transmitted wirelessly or by wires to the device 10 for analysis (Step 100). The algorithm may filter the amplified signal into a readable form and display it for the user on a display. The signal information may then be organized and recorded into identifiable indices. For example, the inter-beat (R-R) interval may be generated from the ECG signal and displayed numerically or visually in the form of a graph, chart, or other visible indicia (Step 102). From the R-R data, an HRV index may then be created for a particular patient (Step 104). This index may be stored and compared to previously recorded HRV indexes to track the overall health of the patient.

The algorithm may then create a correlation coefficient (r) (Step 106) and a coefficient of determination (r2) (Step 108) between heart rate generation (HR) and HRV as discussed in more detail below. From the correlation coefficient and the coefficient of determination, HRV variables can be dissociated from the R-R data and determined for each patient. Additional patient information, such as respiratory rate and blood pressure, may also be analyzed and integrated with the ECG data for accurate real time monitoring of a patient. The measurement of HRV, from signal acquisition to calculating and measuring the HRV may be accomplished in approximately five minutes or less, allowing for faster examinations and minimizing patient discomfort.

For example, in an exemplary method of operation, baseline ECG measurements may be recorded for approximately five minutes while a patient is in a supine position. Next, approximately three minutes of a variety of cardiovascular function tests may be performed. For example, a first test may require the patient to stand from the supine position and the ECG will be recorded for approximately four minutes while the patient remains standing. A second performed test may be the Valsalva maneuver, wherein the patent takes a large inspiration following by a maximum expiratory effort against an obstruction, all while the ECG is recorded. Lastly, a deep breathing exercise may performed while the ECG is recorded, wherein the patients breaths at a rate of six breaths per minute for a period of one minute. Some or all these tests may be performed within five minutes depending on the capabilities of the patient.

The HRV variables calculated from the ECG may be then categorized as either time domain or frequency variables. Time domain variables may include the standard deviation of normal R-R intervals over the recording period (SDNN), the root mean squared of the successive differences (RMSSD), and/or coefficient of variation (COV). Frequency domain variables may include total power (TP) in the frequency range from 0.01 to 0.04 Hz, which may be a reflection of the parasympathetic and sympathetic system, high frequency (HF) power in the frequency of 0.15 to 0.4 Hz, low frequency (LF) power in the frequency range of 0.04 to 0.15 Hz, very low frequency (VLF) in the range of 0.01 to 0.04 Hz, and/or the ratio to LF to HF.

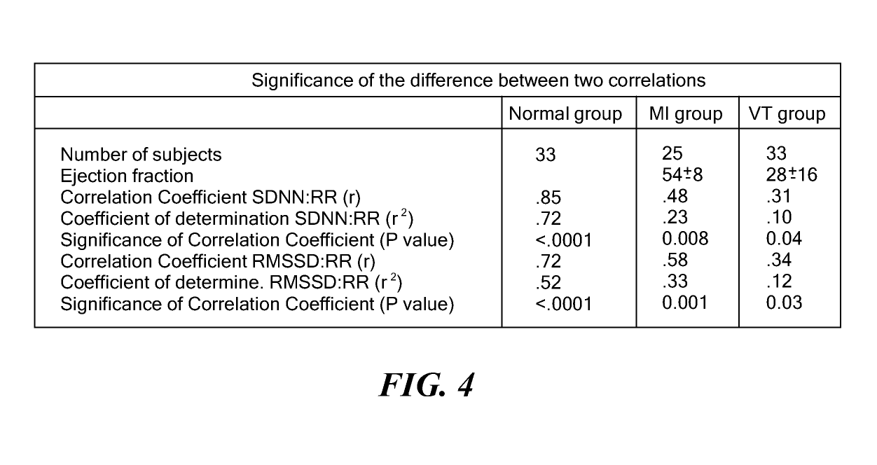

Dissociating the foregoing time domain variables from the measured R-R data, and correlating that data with the calculated HRV during recording period may show that the relationship between the time domain variables of HRV and HR may identify healthy patients from patients with coronary disorders. For example, as shown in FIG. 4, R-R intervals may be recorded for five minutes and analyzed from the three sampling groups; a normal group of heart healthy patients (n=33); a group of patients with chronic coronary artery disease (MI) (n=25); and a group of patients receiving ICDs with a history of ventricular tachycardia (VT) (n=33). The correlation coefficient (r) and coefficient of determination (r²) for the ration of SDNN:R-R may be significantly higher for hearth healthy patients when compared MI and VT patients. Similarly r and r² data for the ratio of RMSSD:R-R may also significantly higher for heart healthy patients when compared to MI and VT patients. This data, for example, may then be indexed and compared each time a patient is tested to determine overall cardiovascular health of the patient.

Clinical information may also be recorded for each patient during testing, which later can be indexed with the patient's HRV data. For example, clinical information may include gender, age, body mass index, systolic blood pressure, diastolic blood pressure, heart rate, class of medications the patient is taking, amount of coffee per day, whether the patient smokes, and/or the patient's medical history.

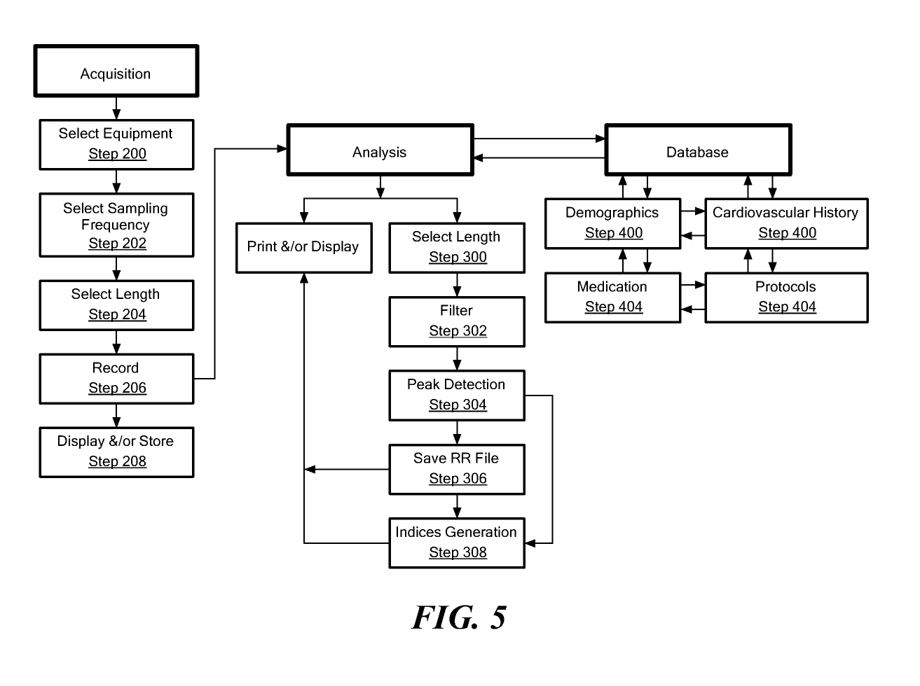

Now referring to FIG. 5, which shows expanded flow chart of the method shown in FIG. 3 including expanded steps for HRV acquisition, analysis, and database indexing. The acquisition method may include the step of selecting the desired equipment (Step 200). This may include selecting the type and number of the at least one biosensor 18 (for example, PPG or ECG biosensors 18) among the other elements of the HRV system discussed above. Once the desired equipment is selected, and the equipment is positioned to sense at least one analog signal 20 from a patient, the next step may be to select the desired sampling frequency (Step 202). For example, R-R intervals may be measured from the ECG signals every millisecond or every half a second, or automatically and pre-set frequencies, for example 250 Hz. Once the desired sampling frequency is selected, the R-R intervals may then be measured for a desired length of time, for example five minutes (Step 204). During ECG acquisition, the R-R interval data may then be amplified and transmitted to the device 10 for digitization, analysis, and recording (Step 206) or displayed, stored, or printed (Step 208).

The analysis of the R-R interval data may include the step of selecting a segment of the R-R interval data for analysis (Step 300). The selected segment of R-R interval data may then be digitally filtered for artifacts, noise, or other outlying data that were recorded during the acquisition step (Step 302). The various peaks may then be detected from the selected segment (Step 304). The time between the peaks, frequency of them, or calculated HRV, may then be recorded or saved (Step 306) in an index created for that particular patient (Step 308).

The analyzed R-R data or HRV data may then be transmitted to a database 40 in a remote location 42, for example, a doctor's office or nurse's station in a hospital. For example, a particular patient's HRV index may be transmitted to a nurse's station at a hospital, where the HRV data may be analyzed against one or more criteria 44, such as the patient's medical history, to predict the likelihood of sudden cardiac arrest or other cardiac related maladies. For example, HRV information for a particular patient may be compared against demographic data applicable to the patient to determine if the patient's HRV is normal for the relevant demographic (Step 400). Additionally, the patient's cardiovascular history, which may include previous HRV data, may be compared against the real-time HRV data collected from the HRV acquisition system (Step 402). It is further contemplated, that in response to the real-time HRV data, a nurse or doctor can create a treatment protocol, which may include medication or medical procedures, to respond to the HRV data (Step 404). The HRV acquisition system may also be utilized to take real time measurements of HRV during the treatment protocol to evaluate the treatment's affect on the patient.

It is further contemplated that the HRV acquisition system can be modified to diagnose and treat non-cardiovascular based conditions. For example, a variety of non-cardiovascular biosensors may be used to detect a number of using the system and method described above. For example, the acquisition system and method may be used to detect, diagnose, and treat, diabetic neuropathy, sleep apnea, depression, the effects of physo-social stress, and other neurological diseases.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A system for measuring heart rate variability (HRV) comprising:
at least one biosensor operable to measure at least one signal from the heart;
a bioamplifier in communication with the at least one biosensor, the bioamplifier being operable to amplify the at least one signal into at least one amplified signal;
a portable device in communication with the bioamplifier, the portable device being in communication an analog to digital converter that digitizes the at least one signal received from the at least one biosensor into one or more digital signals; the portable device being further operable to measure the inter-beat intervals from the one or more digital signals and to calculate HRV from the measured inter-beat intervals; and
a database in communication with the portable device, wherein the calculated HRV is indexed in the database by one or more criteria.

2. The system of claim 1, wherein the at least one signal includes a first signal, wherein the first signal comprises electrical activity.

3. The system of claim 2, wherein the at least one signal includes a second signal, wherein the second signal comprises volumetric measurements.

4. The system of claim 3, wherein the bioamplifier includes a first input in communication with the first signal and a second input in communication with the second signal.

5. The system of claim 4, wherein the analog to digital converter digitizes the first signal into a first digital signal and the second signal into a second digital signal.

6. The system of claim 4, wherein the bioamplifier includes a first output in communication with the first signal and a second output in communication with the second signal.

7. The system of claim 6, wherein the first output transmits a first amplified signal and the second output transmits a second amplified signal.

8. The system of claim 7, further comprising a first wireless transceiver disposed within the portable device, the first wireless transceiver in communication with the first output and the second output.

9. The system of claim 8, wherein the bioamplifier and the at least one biosensor are in communication through a second wireless transceiver coupled to the at least one biosensor.

10. The system of claim 9, further comprising a third wireless transceiver disposed within the bioamplifier, wherein the third wireless transceiver transmits the first amplified signal and the second amplified signal to the portable device.

11. A method for measuring heart rate variability (HRV) comprising:
measuring at least one signal proximate the heart;
amplifying the at least one signal into at least one amplified signal;
digitizing the at least one amplified signal into one or more digital signals;
measuring the inter-beat intervals from the one or more digital signals;
calculating HRV from the inter-beat intervals; and
correlating the measured inter-beat intervals and the calculated HRV to a condition of cardiovascular health.

12. The method of claim 11, further comprising creating a treatment protocol in response to the results of the correlation.

13. The method of claim 11, further comprising indexing the correlated data by one or more criteria.

14. The method of claim 13, storing the indexed data in a remote location.

15. The method of claim 13, wherein the one or more criteria include a patient's medical history.

16. The method of claim 11, wherein measuring the at least one analog signal includes positioning at least one biosensor proximate the heart.

17. The method of claim 16, wherein the at least one biosensor measures electrical activity proximate the heart.

18. The method of claim 16, wherein the at least one biosensor measure changes in volume proximate the heart.

19. method of claim 11, further including wirelessly transmitting the one or more digital signals to a portable device.

20. A method for measuring heart rate variability (HRV) comprising:
providing a first biosensor operable to measure electrical activity proximate the heart; a second biosensor operable to measure changes in volume proximate the heart; a bioamplifier in communication with the first biosensor and the second biosensor; a portable device in communication with the bioamplifier, the portable device being operable to measure the inter-beat intervals and calculate HRV from the measured inter-beat intervals;
positioning the first biosensor and the second biosensor proximate the heart;
measuring a first signal from the first biosensor and a second signal from the second biosensor;
amplifying the first signal and the second signal into a first amplified signal and a second amplified signal;
transmitting the first amplified signal and the second amplified signal to the portable device;
digitizing the first amplified signal to a first digital signal and the second amplified signal to a second digital signal;
measuring the inter-beat interval from the first digital signal or the second digital signal;
calculating HRV from the measured inter-beat interval;
correlating the measured inter-beat intervals and the calculated HRV to a condition of cardiovascular health
indexing the calculated HRV in a remote database by one or more criteria; and
creating a treatment protocol in response to the calculated HRV.
